(19) United States
(12) Patent Application Publication
(10) Pub. No.: US 2026/0308230 A1
(43) Pub. Date: Oct. 8, 2026

(54) RECOMBINANT FUSION PROTEIN FOR TREATMENT OF PULMONARY HYPERTENSION

(71) Applicant: IMMUNECARE BIOPHARMACEUTICALS (SHANGHAI) CO., LTD, Shanghai (CN)

(72) Inventors: Wenzhi TIAN, Shanghai (CN); Song LI, Shanghai (CN); Dianze CHEN, Shanghai (CN)

(21) Appl. No.: 19/482,303

(22) PCT Filed: Apr. 8, 2024
(86) PCT No.: PCT/CN2024/086633

§ 371 Date: Nov. 7, 2025

(30) Foreign Application Priority Data
May 15, 2023 (WO) PCT/CN2023/094262

(51) Int. Cl. C07K 14/71; A61K 38/00; A61P 9/14

(52) U.S. Cl. CPC C07K 14/71; A61P 9/14; A61K 38/00; C07K 2319/30

(57) ABSTRACT

Provided is a recombinant fusion protein which contains an extracellular domain of human activin receptor type 2A fused, optionally with a linker, to an immunoglobulin heavy chain constant region, wherein the extracellular domain of the human ACVR2A comprises an Asn (N)-to-Ala (A) mutation at a site corresponding to position 24 of the sequence set forth in SEQ ID NO: 1 to remove a glycosylation site.

### FIELD OF THE INVENTION

The application relates to a recombinant fusion protein that contains an extracellular domain of human activin receptor type 2A (ACVR2A) fused to an immunoglobulin heavy chain constant region, wherein the extracellular domain of the human ACVR2A comprises a mutation at a site corresponding to position 24 of the amino acid sequence of SEQ ID NO: 1 which removes a glycosylation site. The application also relates to the use of the recombinant fusion protein of the disclosure in treatment of pulmonary hypertension and associated complications.

### BACKGROUND OF THE INVENTION

Pulmonary hypertension (PH) is a chronic and progressive disease characterized by increased pressure in the pulmonary arteries, leading to right heart failure and ultimately death if left untreated. PH can be idiopathic or associated with various underlying conditions such as connective tissue diseases, congenital heart disease, and chronic lung disease. The symptoms of PH include shortness of breath, fatigue, chest pain, and syncope.

PH is diagnosed through a combination of physical examination, imaging tests such as echocardiography, right heart catheterization, and blood tests to assess oxygen levels and other markers of disease severity. Treatment for PH depends on the underlying cause but often includes medications to dilate the pulmonary arteries and reduce pressure, supplemental oxygen, and lifestyle modifications.

Sotatercept, a fusion protein used in treating PH, comprises an extracellular domain of a wild type activin receptor type 2A (ACVR2A) fused to an Fc domain of an immunoglobulin. It traps activins and growth differentiation factors involved in pulmonary hypertension and thus inhibits signaling pathways that lead to abnormal growth and constriction of pulmonary blood vessels, reducing the workload on the right side of the heart and improving blood flow to the lungs.

Clinical studies have shown that treatment with sotatercept can significantly improve exercise capacity, reduce pulmonary vascular resistance, and improve hemodynamic parameters in patients with PH. Sotatercept is currently undergoing further clinical trials to evaluate its long-term safety and efficacy in PAH and other pulmonary vascular disorders.

WO2021262718A1 discloses compositions comprising the ACVR2A-Fc fusion proteins (e.g., sotatercept) and methods for treating or preventing PH and PH associated complications using the same.

Citation or identification of any document in this application is not an admission that such a document is available as prior art to the present disclosure.

### SUMMARY OF THE INVENTION

The inventors of the present application have designed and prepared a recombinant fusion protein comprising an extracellular domain of human activin receptor type 2A (ACVR2A) fused to an immunoglobulin heavy chain constant region, which comprises a de-glycosylation mutation in the extracellular domain of human ACVR2A at a site corresponding to the position 24 of SEQ IN NO: 1, and is superior than sotatercept in treating or alleviating pulmonary hypertension and associated complications. Particularly, the administration of the recombinant fusion protein of the present application can further improve animal survival as compared to sotatercept in the monocrotaline induced pulmonary arterial hypertension model.

Accordingly, in a first aspect, the disclosure provides a recombinant fusion protein which may comprise i) an extracellular domain of an activin receptor type 2A (ACVR2A) linked to ii) an immunoglobulin heavy chain constant region, wherein the extracellular domain of the ACVR2A may comprise a asparagine (Asn, N) to alanine (Ala, A) mutation at a site corresponding to the position 24 of the amino acid sequence of SEQ ID NO: 1 and is able to bind activins or growth differentiation factors, especially with high affinity/capability. The extracellular domain of the ACVR2A may be able to bind activins and growth differentiation factors, especially with high affinity/capability. In certain embodiments, the extracellular domain of the ACVR2A may be able to bind activins and growth differentiation factors with high affinity/capability. The extracellular domain of the ACVR2A may comprise asparagine (Asn, N) or alanine (Ala, A) at a site corresponding to the position 47 of the amino acid sequence of SEQ ID NO: 1. In certain embodiments, the extracellular domain of the ACVR2A may comprise asparagine (Asn, N) at a site corresponding to position 47 of the amino acid sequence of SEQ ID NO: 1.

The activin receptor type 2A may be a human activin receptor type 2A. The extracellular domain of the human activin receptor type 2A may comprise a asparagine (Asn, N) to alanine (Ala, A) mutation at a site corresponding to the position 24 of the amino acid sequence of SEQ ID NO: 1. In certain embodiments, the extracellular domain of the ACVR2A may comprise asparagine (Asn, N) at a site corresponding to the position 47 of the amino acid sequence of SEQ ID NO: 1. The extracellular domain of the human activin receptor type 2A may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 1 (X1=A, X2=N). In certain embodiments, the extracellular domain of the human activin receptor type 2A may comprise an amino acid sequence having at least 95% sequence identity to SEQ ID NO: 1 (X1=A, X2=N). In certain embodiments, the extracellular domain of the human activin receptor type 2A may comprise the amino acid sequence of SEQ ID NO: 1 (X1=A, X2=N).

The immunoglobulin heavy chain constant region may be a human immunoglobulin heavy chain constant region, e.g., human IgG1 or IgG4 heavy chain constant region. The immunoglobulin heavy chain constant region may be an Fc domain of an immunoglobulin heavy chain constant region. In certain embodiments, the immunoglobulin heavy chain constant region may be an Fc domain of human IgG1 heavy chain constant region, comprising the amino acid sequence of e.g., SEQ ID NO: 2.

The recombinant fusion protein of the disclosure may comprise, from the N-terminus to the C-terminus, the extracellular domain of human ACVR2A and the immunoglobulin heavy chain constant region.

The extracellular domain of the activin receptor type 2A may be fused to the immunoglobulin heavy chain constant region via a linker. The linker may be a peptide of about 5 to 30 amino acid residues. In certain embodiments, the linker may be a peptide of about 5 to 20 amino acid residues. In certain embodiments, the linker may be a GS linker comprising the amino acid sequence of e.g., SEQ ID NO: 3. The recombinant fusion protein of the disclosure may comprise an extracellular domain of human activin receptor type 2A fused via a linker to an Fc domain of human immunoglobulin heavy chain constant region, wherein the extracellular domain of the human activin receptor type 2A may comprise a asparagine (Asn, N) to alanine (Ala, A) mutation at a site corresponding to the position 24 of the amino acid sequence of SEQ ID NO: 1. In certain embodiments, the recombinant fusion protein of the disclosure may comprise, from N-terminus to C-terminus, the extracellular domain of human activin receptor type 2A, the linker and the Fc domain of human immunoglobulin heavy chain constant region. The extracellular domain of the human activin receptor type 2A may comprise asparagine (Asn, N) at a site corresponding to the position 47 of the amino acid sequence of SEQ ID NO: 1. In certain embodiments, the recombinant fusion protein of the disclosure may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 4 (X1=A, X2=N). In certain embodiments, the recombinant fusion protein of the disclosure may comprise the amino acid sequence of SEQ ID NO: 4 (X1=A, X2=N).

The disclosure also provides a dimer comprising the recombinant fusion protein of the disclosure. In certain embodiments, the disclosure provides a homodimer of the recombinant fusion protein of the disclosure, comprising two recombinant fusion proteins of the disclosure linked via e.g., by one or more disulfide bonds.

The recombinant fusion protein or the dimer (including the homodimer) of the disclosure may bind to one or more ligands selected from the group consisting of activin A, activin B, BMP-10, GDF-8 and GDF11, of e.g., human origin, and may block interaction of activin A, activin B, BMP-10, GDF-8 or GDF11 to the human activin receptor type 2A.

In a second aspect, the disclosure provides a nucleic acid molecule encoding the recombinant fusion protein of the disclosure, as well as an expression vector that may comprise the nucleic acid molecule and a host cell that may comprise the expression vector or have the nucleic acid molecule integrated in its genome. A method for preparing the recombinant fusion protein or the homodimer of the disclosure using the host cell is also provided, that may comprise steps of (i) expressing the recombinant fusion protein or the homodimer in the host cell and (ii) isolating the recombinant fusion protein or the homodimer from the host cell or its cell culture.

The disclosure provides a composition that may comprise the recombinant fusion protein, the dimer (including the homodimer), the nucleic acid molecule, the expression vector, or the host cell of the disclosure. In certain embodiments, the composition is a pharmaceutical composition, which may further a pharmaceutically acceptable carrier.

The disclosure also provides a kit that may comprise a recombinant fusion protein or a dimer (including the homodimer) of the disclosure, and an injection device. The recombinant fusion protein or the dimer (including the homodimer) of the disclosure may be lyophilized or soluble. In certain embodiments, the recombinant fusion protein or the dimer (including the homodimer) is lyophilized.

In a third aspect, the disclosure provides a method for treating or alleviating pulmonary hypertension (PH) and/or associated complications in a subject in need thereof, comprising administering to the subject a therapeutically effective amount of the pharmaceutical composition of the disclosure. The associated complications may include, but not limited to, smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis. In certain embodiments, the subject is human.

The disclosure also provides the use of the pharmaceutical composition of the disclosure in treating or alleviating pulmonary hypertension and/or associated complications, and in the preparation of a medicament for treating or alleviating pulmonary hypertension and/or associated complications.

Other features and advantages of the instant disclosure will be apparent from the following detailed description and examples which should not be construed as limiting. The contents of all references, GenBank entries, patents and published patent applications cited throughout this application are expressly incorporated herein by reference.

Accordingly, it is an object of the application not to encompass within the application any previously known product, process of making the product, or method of using the product such that Applicants reserve the right and hereby disclose a disclaimer of any previously known product, process, or method. It is further noted that the application does not intend to encompass within the scope of the application any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the application to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the application.

### DESCRIPTION OF THE DRAWINGS

The following detailed description, given by way of example, but not intended to limit the application solely to the specific embodiments described, may best be understood in conjunction with the accompanying drawings.

FIG. 1 shows the SEC-HPLC results of the sotatercept analogue (A), ACVR2A-Fc (B), ACVR2A-24A-Fc (IMM72, C), ACVR2A-47A-Fc (D) and ACVR2A-24A/47A-Fc (E).

FIG. 2 shows the SDS-PAGE gel image of the ACVR2A-Fc fusion proteins, including the sotatercept analogue (lane 1), ACVR2A-Fc (lane 2), ACVR2A-24A-Fc (IMM72, lane 3), ACVR2A-47A-Fc (lane 4) and ACVR2A-24A/47A-Fc (lane 5).

FIG. 3 shows the BLI binding curves of ACVR2A-Fc (A), ACVR2A-24A-Fc (IMM72, B), ACVR2A-47A-Fc (C) and ACVR2A-24A/47A-Fc (D) to the activin A.

FIG. 4 shows BLI binding curves of the sotatercept analogue (A) and IMM72 (B) to the activin A.

FIG. 5 shows the binding capability of the ACVR2A-Fc fusion proteins to the activin A as measured in ELISA.

FIG. 6 shows the K_{D} values (A) and shifts (B) in BLI tests measuring the ACVR2A-Fc fusion proteins' binding affinity to 5 ligands.

FIG. 7 shows the capability of the ACVR2A-Fc fusion proteins to block ACVR2A's binding or interaction with activin A (A), activing B (B) or BMP-10 (C) in a cell-based assay.

FIG. 8 shows the capability of the ACVR2A-Fc fusion proteins to block ACVR2A's binding or interaction with activin A (A), activing B (B), GDF-11 (C), BMP-10 (D) or GDF-8 (E) in a cell-based assay.

FIG. 9 shows the survival rate of rats treated with monocrotaline and the ACVR2A-Fc fusion proteins.

FIG. 10 shows the PAAT/PET ratio (A) and RVH index (B) of rats treated with monocrotaline and ACVR2A-Fc fusion proteins.

### DETAILED DESCRIPTION OF THE APPLICATION

To ensure that the present disclosure may be more readily understood, certain terms are first defined. Additional definitions are set forth throughout the detailed description.

The term “ACVR2A” refers to activin receptor type 2A. The term “ACVR2A” comprises variants, isoforms, homologs, orthologs and paralogs. The “variant” or “mutant” of ACVR2A or its extracellular domain refers to an ACVR2A protein or an ACVR2A extracellular domain that contains one or more mutations relative to the wildtype ACVR2A protein or the wildtype ACVR2A extracellular domain. For example, an ACVR2A variant may contain a mutation in the extracellular domain that corresponds to asparagine (Asn, N) to alanine (Ala, A) at the position 24 of the amino acid sequence of SEQ ID NO: 1.

The term “immunoglobulin”, also referred to as an antibody, refers to a molecule produced by white blood cells that help the body defend against infections and cancers. There are five types of immunoglobulins, i.e., IgG, IgM, IgA, IgD and IgE. A whole or full-length antibody comprises at least two heavy (H) chains and two light (L) chains inter-connected by disulfide bonds. There are five types of mammalian Ig heavy chains denoted by Greek letters: α, δ, ε, γ and μ. These chains are found in IgA, IgD, IgE, IgG, and IgM antibodies, respectively. Each heavy chain is comprised of a heavy chain variable region (abbreviated herein as V_{H}) and a heavy chain constant region (C_{H}). The heavy chain constant region is identical in all the same isotype antibodies but differs in antibodies of different isotypes. Heavy chains γ, α, and δ have a constant region composed of three tandem Ig domains—C_{H1}, C_{H2}, C_{H3}—and a hinge region for added flexibility. Heavy chains μ and ε have a constant region composed of four immunoglobulin domains. The heavy chain constant region of the antibody may mediate the binding of the immunoglobulin to host tissues or factors, including various cells of the immune system (e.g., effector cells) and the first component (C1q) of the classical complement system.

The term “Fc domain” of an immunoglobulin or an antibody is the tail region of an antibody that interacts with Fc receptors and some proteins of the complement system to activate the immune system. The IgG, IgA and IgD Fc domain is composed of two identical fragments derived from the second and third constant domains (C_{H2} and C_{H3}) of the antibody's heavy chains, while the IgM and IgE Fc regions contain three heavy chain constant domains (C_{H} domains 2-4).

The term “EC₅₀”, also known as half maximal effective concentration, refers to the concentration of a molecule, e.g., a recombinant fusion protein or a dimer of the disclosure, which induces a response halfway between the baseline and maximum after a specified exposure time.

The term “IC₅₀”, also known as half maximal inhibitory concentration, refers to the concentration of a molecule, e.g., a recombinant fusion protein or a dimer of the disclosure, which inhibits a specific biological or biochemical function by 50% relative to the absence of the molecule.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using the publicly available computer software such as ClustalOmega, T-coffee, Kalign and MAFFT. When using such softwares, the default parameters, e.g., for gap penalty and extension penalty, are preferably used.

The term “therapeutically effective amount” means an amount of a molecule, e.g., a recombinant fusion protein or a dimer of the present disclosure sufficient to prevent or ameliorate the symptoms associated with a disease or condition (such as pulmonary hypertension) and/or lessen the severity of the disease or condition. A therapeutically effective amount is understood to be in context to the condition being treated, where the actual effective amount is readily discerned by those of skill in the art.

The term “with high affinity/capability” herein means the recombinant fusion protein of the disclosure has a binding affinity, activity or capability that is comparable to or higher than the sotatercept or the wildtype ACVR2A.

Pulmonary hypertension (PH) is a type of high blood pressure that affects the arteries in the right side of the heart and the lungs. Pulmonary arterial hypertension (PAH) is one form of PH characterized by pulmonary vascular remodeling that is driven by enhanced proliferation or reduced apoptosis of endothelial and smooth-muscle cells, causing the narrowing of or damage to the blood vessels in the lungs.

The bone morphogenetic protein receptor type 2 (BMPR2), a member of the transforming growth factor β (TGF-β) superfamily, plays an important role in maintaining endothelial integrity of pulmonary arteries, while mutations in this protein may lead to PAH. Sotatercept functions as a ligand trap for TGF-β superfamily members, including activins and bone morphogenetic proteins, restoring the balance between growth-promoting and growth-inhibiting signaling pathways.

The inventors of the disclosure have designed and prepared a recombinant fusion protein comprising an extracellular domain of human activin receptor type 2A (ACVR2A) fused to an immunoglobulin heavy chain constant region, which differs from sotatercept in the glycosylation site in the extracellular domain of the human activin receptor 2A located at a site that corresponds to the position 24 of the amino acid sequence of SEQ ID NO: 1. In particular, the recombinant fusion protein of the disclosure lacks a glycosylation site in the extracellular domain of the human activin receptor type 2A located at a site that corresponds to the position 24 of the amino acid sequence of SEQ ID NO: 1. With the de-glycosylation mutation at the specified site, the recombinant fusion protein of the disclosure shows higher response in the activin A, activin B, BMP-10, GDF-11 or GDF-8 binding affinity tests than the sotatercept analogue. That is, the response at the equilibrium seems to be higher for the recombinant fusion protein of the disclosure, i.e., more ligand molecules might bind to the recombinant fusion protein of the disclosure as compared to the sotatercept analogue (see Example 3 and Example 5). The recombinant fusion protein of the disclosure also shows much higher binding capability to activin A than the sotatercept analogue and ACVR2A (WT)-Fc protein in an ELISA (see Example 4), Further, the recombinant fusion protein of the disclosure shows higher capability to inhibit ACVR2A-activin A interaction, and lower IC₅₀ values in blocking ACVR2A-activin B/GDF11/BMP-10 interaction, than the ACVR2A (WT)-Fc protein and the sotatercept analogue (see Example 6).

Most importantly, the recombinant fusion protein of the disclosure is superior than sotatercept in treating or alleviating pulmonary hypertension and associated complications. Specifically, the administration of the recombinant fusion protein of the disclosure is more efficacious in alleviating right ventricular hypertrophy, thus improving animal survival, as compared to sotatercept, in the monocrotaline induced pulmonary arterial hypertension model.

It is well known in the field of protein engineering that a de-glycosylation mutation may alter the biological activity of a protein in unpredictable manners.

Wang et al discloses N-linked glycosylation of certain amino acid residues in the PD-L1 molecule plays an important role in the PD-L1's binding affinity to PD-1 and its immune-suppressive function in vivo. De-glycosylation promoted PD-L1 degradation and inhibits PD-L1 binding to PD-1, and the removal of N-linked glycosylation in the PD-L1 molecule in fixed tissue samples enhanced antibody binding.

Tansky et al reveals that although complete removal of N-linked glycosylation significantly reduced NK1R's Bₘₐₓ in vitro, its binding affinity to substance P was not affected. Further, the de-glycosylation did not have a profound effect on the NK1R' ability to activate the MAP kinase families (p42/p44, JNK, and p38), but did affect SP-induced IL-8 secretion.

The extracellular domain of activin receptor type 2A contains at least two putative N-linked glycosylation sites that correspond to the position 24 and position 47 of the amino acid sequence of SEQ ID NO: 1. The de-glycosylation at these two sites surprisingly resulted in different molecule homogeneities and activity.

In particular, the inventors of the disclosure constructed three recombinant fusion protein dimers each containing a mutant extracellular domain of activin receptor type 2A, namely ACVR2A-24A-Fc (or IMM72), ACVR2A-47A-Fc, and ACVR2A-24A/47A-Fc. IMM72, ACVR2A-47A-Fc and ACVR2A-24A/47A-Fc are otherwise identical, except that IMM72 contains N24A de-glycosylation mutation, while ACVR2A-47A-Fc contains N47A de-glycosylation mutation and ACVR2A-24A/47A-Fc contains both N24A and N47A de-glycosylation mutations, wherein the numbering of amino acid residues for these fusion proteins is based on the numbering of SEQ ID NO: 1. Surprisingly, as revealed in the Examples below, the protein homogeneity (as demonstrated in the SEC-HPLC and SDS-PAGE data) and biological activity of IMM72 (as shown in the Grato affinity and ELISA binding data) are dramatically better than those of ACVR2A-47A-Fc or ACVR2A-24A/47A-Fc.

Even more surprisingly, the protein homogeneity and in vivo efficacy of IMM72, with the N to A mutation at a site corresponding to the position 24 of SEQ ID NO: 1, is even better than that of sotatercept which utilizes the wildtype ACVR2A extracellular domain.

The recombinant fusion protein of the disclosure comprises i) an extracellular domain of activin receptor type 2A, ii) an optional liker, and iii) an immunoglobulin heavy chain constant region.

The activin receptor type 2A may be a human activin receptor type 2A. The extracellular domain of the human activin receptor type 2A may comprise a mutation that may correspond to asparagine (Asn, N) to alanine (Ala, A) at the position 24 of the amino acid sequence of SEQ ID NO: 1. The extracellular domain of the ACVR2A may comprise asparagine (Asn, N) at a site corresponding to the position 47 of the amino acid sequence of SEQ ID NO: 1. The extracellular domain of the human activin receptor type 2A may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 1 (X1=A, X2=N). In certain embodiments, the extracellular domain of the human activin receptor type 2A may comprise an amino acid sequence having at least 95% sequence identity to SEQ ID NO: 1 (X1=A, X2=N). In certain embodiments, the extracellular domain of the human activin receptor type 2A may comprise the amino acid sequence of SEQ ID NO: 1 (X1=A, X2=N).

N-linked glycosylation occurs at the consensus sequence Asn-X-Ser/Thr, where the glycan attaches to the amine group of asparagine and X represents any amino acid except proline. Any changes to the consensus sequence may lead to de-glycosylation. In other words, the mutation may apply to any one of the amino acid residues located at the sites corresponding to the position 24 to 26 of SEQ ID NO: 1, as long as the mutation can lead to de-glycosylation. For example, asparagine (Asn, N) at the site corresponding to the position 24 of SEQ ID NO: 1 may be deleted or replaced with another amino acid to remove the glycosylation site. The amino acid residue at the site corresponding to the position 25 of SEQ ID NO: 1 may be deleted or replaced with proline (Pro, P). Alternatively, serine (Ser, S) or threonine (Thr, T) at the site corresponding to the position 26 of SEQ ID NO: 1 may be deleted or replaced with an amino acid residue other than Ser and Thr.

The extracellular domain of activin receptor type 2A may comprise one or more conservative modifications. It is understood in the art that certain conservative sequence modification can be made which does not remove a protein's binding capability to e.g., a ligand. The extracellular domain of activin receptor type 2A of the disclosure, with or without the conservative modification(s), may bind to one or more ligands selected from the group consisting of activin A, activin B and GDF11. The extracellular domain of activin receptor type 2A of the disclosure, with or without the conservative modification(s), may further bind to one or more ligands selected from the group consisting of BMP10, GDF8 and BMP6.

As used herein, the term “conservative sequence modification” is intended to refer to amino acid modifications that do not significantly affect or alter the binding characteristics of the ACVR2A extracellular domain containing the amino acid sequence. Such conservative modifications include amino acid substitutions, additions and deletions. Modifications can be introduced into the ACVR2A extracellular domain of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the ACVR2A extracellular domain of the disclosure can be replaced with other amino acid residues from the same side chain family and the altered extracellular domain can be tested for retained function, e.g., the binding capability to e.g., activin A, activin B, GDF11, BMP10, GDF8 and/or BMP6, using the functional assays described herein.

The immunoglobulin heavy chain constant region is mainly used to stabilize the structure of the recombinant fusion protein of the disclosure, and/or to prolong the serum half-life of the recombinant fusion protein of the disclosure. A full-length heavy chain constant region or a fragment thereof, such as an Fc domain, may be used, as long it can stabilize the structure and/or to prolong the serum half-life of the recombinant fusion protein of the disclosure.

The immunoglobulin heavy chain constant region may be a human immunoglobulin heavy chain constant region, e.g., human IgG1 or IgG4 heavy chain constant region. The immunoglobulin heavy chain constant region may be an Fc domain of an immunoglobulin heavy chain constant region. In certain embodiments, the immunoglobulin heavy chain constant region may be an Fc domain of human IgG1 heavy chain constant region, comprising the amino acid sequence of e.g., SEQ ID NO: 2.

The extracellular domain of the activin receptor type 2A may be fused to the immunoglobulin heavy chain constant region via a linker.

The linker may be made up of amino acids linked together by peptide bonds, preferably from 5 to 30 amino acids linked by peptide bonds, wherein the amino acids are selected from the 20 naturally occurring amino acids. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines, particularly poly(Gly-Ala), and polyalanines. In certain embodiments, the linker may be a peptide of about 5 to 20 amino acid residues. In certain embodiments, the linker may be a GS linker comprising the amino acid sequence of e.g., SEQ ID NO: 3.

The linker may also be a non-peptide linker. For example, alkyl linkers such as —NH—, —(C_{H2})s-C(O)—, wherein s=2-20 can be used. These alkyl linkers may further be substituted by any non-sterically hindering group such as lower alkyl (e.g., C₁₋₆) lower acyl, halogen (e.g., CI, Br), CN, NH₂, phenyl, etc.

The linker peptide selection may alter the biological activity of a protein. The linker peptide length, flexibility, composition and context may have significant impact on fusion protein activity. For example, Silacci et al. discloses that longer linker length corresponds to higher activity for a peptide inhibitor of IL-17A. However, conversely, Guo et al reveals that catalytic efficiency of a fusion protein enzyme decreased gradually with the increase of GS repeats.

In another aspect, the disclosure provides a nucleic acid molecule that encodes the recombinant fusion protein or the dimer (including the homodimer) of the disclosure.

The nucleic acid molecule can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

The nucleic acid molecule of the disclosure can be obtained using standard molecular biology techniques. For example, the nucleic acid molecule may be chemically synthesized. Alternatively, the nucleic acid molecule may be obtained by making mutations in a nucleic acid molecule encoding the extracellular domain of a wildtype activin receptor type 2A fused to an immunoglobulin heavy chain constant region via e.g., site-directed mutagenesis or PCR-mediated mutagenesis. A DNA fragment encoding the ACVR2A extracellular domain may be operatively linked to a DNA fragment encoding the immunoglobulin heavy chain constant region. The term “operatively linked”, as used in this context, is intended to mean that the two DNA fragments are joined such that the amino acid sequences encoded by the two DNA fragments remain in-frame.

The recombinant fusion protein or the dimer of the disclosure may be produced by i) inserting the nucleotide sequences encoding the recombinant fusion protein into an expression vector which is operatively linked to regulatory sequences that control transcription or translation; (ii) transducing or transfecting host cells with the expression vectors; and (iii) expressing the recombinant fusion protein of the disclosure and forming the dimer of the disclosure.

The term “regulatory sequence” is intended to include promoters, enhancers and other expression control elements (e.g., polyadenylation signals) that control the transcription or translation of the nucleic acid molecules of the disclosure.

The expression vector can encode a signal peptide that facilitates secretion of the recombinant fusion proteins from a host cell. The nucleic acid molecule of the disclosure can be cloned into the vector such that the signal peptide is linked in-frame to the amino terminus of the recombinant fusion protein of the disclosure.

The expression vectors of the disclosure can carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

The expression vector(s) can be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the recombinant fusion protein of the disclosure in either prokaryotic or eukaryotic host cells, expression of the recombinant fusion protein in eukaryotic cells, and most preferably mammalian host cells, is the most preferred because such eukaryotic cells, and in particular mammalian cells, are more likely than prokaryotic cells to assemble and secrete a properly folded and immunologically active molecule.

The expression vectors that can be used in the present application include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs).

In another aspect, the present disclosure provides a pharmaceutical composition which may comprise the recombinant fusion protein or the dimer, the nucleic acid molecule, the expression vector, or the host cell, of the disclosure, formulated together with a pharmaceutically acceptable carrier. The pharmaceutical composition may optionally contain one or more additional pharmaceutically active ingredients, as needed, such as an additional pharmaceutically active ingredient effective in treating or alleviating pulmonary hypertension and associated complications. The pharmaceutical composition of the disclosure may be used in combination with an additional pharmaceutically active ingredient effective in treating or alleviating pulmonary hypertension and associated complications.

The pharmaceutical composition may comprise any number of excipients. Excipients that can be used include carriers, surfactants, thickening or emulsifying agents, solid binders, dispersion or suspension aids, solubilizers, colorants, flavoring agents, coatings, disintegrating agents, lubricants, sweeteners, preservatives, isotonic agents, and combinations thereof.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, the pharmaceutical composition of the disclosure can be administered via a non-parenteral route, such as a topical, epidermal or mucosal route of administration, e.g., intranasally, orally, vaginally, rectally, sublingually or topically.

The pharmaceutical compositions can be in the form of a sterile aqueous solution or dispersion. It can also be formulated in a micro-emulsion, liposome, or other ordered structure suitable to high drug concentration.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid.

The administration of the pharmaceutical composition of the disclosure may be determined by physicians, e.g., doctors, depending on a subject's e.g., sex, age, medical history and the like.

A “therapeutically effective dosage” of the pharmaceutical composition of the disclosure, may result in a decrease in severity of disease symptoms, or an increase in frequency and duration of disease symptom-free periods. For example, for the treatment of a subject having pulmonary hypertension and associated complications, a “therapeutically effective dosage” may increase the PAAT/PET ratio by at least about 20%, more preferably by at least about 40%, even more preferably by at least about 60%, and still more preferably by at least about 80%, or alternatively decreased the index of right ventricular hypertrophy by at least about 20%, more preferably by at least about 40%, even more preferably by at least about 60%, and still more preferably by at least about 80%, relative to untreated subjects.

### Uses and Methods

The pharmaceutical composition of the disclosure may be used to treat or alleviate pulmonary hypertension and associated complications.

The disclosure provides methods of combination therapy in which the pharmaceutical composition of the present disclosure is co-administered with one or more additional agents that are effective in treatment or alleviation of pulmonary hypertension and associated complications.

In one embodiment, the disclosure provides a method for treating pulmonary arterial hypertension (PAH) in a subject in need thereof, comprising administering to the subject a therapeutically effective amount of the pharmaceutical composition of the disclosure. The administration of the pharmaceutical composition may result in a change in one or more of the following hemodynamic or functional parameters: a reduction in pulmonary vascular resistance (PVR); an increase in 6-minute walk distance (6MWD); a decrease of the N-terminal pro B-type natriuretic peptide (NT-proBNP) levels; the prevention or reduction of pulmonary hypertension Functional Class progression as recognized by the World Health Organization (WHO); the promotion or increasing of pulmonary hypertension Functional Class regression as recognized by the WHO; an improvement in right ventricular function; an improvement in pulmonary artery pressure; and/or an improvement in mean right atrial pressure.

In another embodiment, the disclosure provides a method for treating, preventing, or reducing the progression rate and/or severity of one or more complications of pulmonary arterial hypertension in a subject in need thereof, comprising administering to the subject a therapeutically effective amount of the pharmaceutical composition of the disclosure. In certain embodiments, the one or more complications of pulmonary arterial hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis.

In another embodiment, the disclosure provides a method for treating or preventing cardiopulmonary remodeling associated with pulmonary arterial hypertension in a subject in need thereof, comprising administering to the subject a therapeutically effective amount of the pharmaceutical composition of the disclosure. The administration of the pharmaceutical composition may slow down cardiac remodeling and/or reverses cardiac remodeling. In some embodiments, the cardiopulmonary remodeling is ventricle remodeling. In some embodiments, the ventricle remodeling is right ventricular remodeling. In some embodiments, the cardiopulmonary remodeling is ventricular dilation.

In certain embodiments, the administration of the pharmaceutical composition of the disclosure may increase the PAAT/PET ratio, decrease the index of right ventricular hypertrophy, and/or improve the patient survival rate.

Various aspects and embodiments of the disclosure will be discussed in reference to the drawings and the following Examples. Other aspects and embodiments are apparent to those skilled in the art. All references described herein are incorporated herein by reference in their entirety. Although the present application has been described in reference to the exemplary embodiments, many changes and alterations, in view of the disclosure, are apparent to those skilled in the art. Therefore, the exemplary embodiments of the disclosure are only provided for illustration, and should not been construed as limiting. Various alterations may be made to the embodiments without departing from the sprit and scope of the application.

### EXAMPLES

IMM72, also referred to as ACVR2A-24A-Fc, is a homodimer of an exemplary recombinant fusion protein of the disclosure comprising the amino acid sequence of SEQ ID NO: 4 (X1=A, X2=N), which recombinant fusion protein comprises a N to A mutation at the position 24 of SEQ ID NO: 1.

ACVR2A-47A-Fc is a homodimer of a recombinant fusion protein comprising the amino acid sequence of SEQ ID NO: 4 (X1=N, X2=A), which recombinant fusion protein comprises a N to A mutation at the position 47 of SEQ ID NO: 1.

ACVR2A-24A/47A-Fc is a homodimer of a recombinant fusion protein comprising the amino acid sequence of SEQ ID NO: 4 (X1=A, X2=A), which recombinant fusion protein comprises a N to A mutation at the position 24 of SEQ ID NO: 1 and a N to A mutation at the position 47 of SEQ ID NO: 1.

ACVR2A-Fc, also referred to as ACVR2A (WT)-Fc, is a homodimer of a recombinant fusion protein comprising the amino acid sequence of SEQ ID NO: 4 (X1=N, X2=N), with the wildtype extracellular domain of human ACVR2A (SEQ ID NO: 1, X1=N, X2=N) fused to an Fc domain of human IgG1 heavy chain constant region.

The sotatercept analogue is a homodimer of a recombinant fusion protein comprising the extracellular domain of a wildtype activin receptor type 2A fused to an Fc domain of human IgG heavy chain constant region, wherein the recombinant fusion protein comprises the amino acid sequence of SEQ ID NO: 5.

### Example 1. Generation and Purification of Recombinant ACVR2A-Fc Fusion Proteins

The DNA fragments encoding the recombinant ACVR2A-Fc fusion proteins, including ACVR2A-24A-Fc, ACVR2A-47A-Fc, ACVR2A-24A/47A-Fc, ACVR2A-Fc and the sotatercept analogue, were synthesized, digested with HindIII and NotI, and ligated into the pMac-Fc vectors having been digested with the same two restriction enzymes, to construct the expression vectors.

CHO-S cells were cultured under TransFx-C CHO Transient transfection medium (Cat #SH30942.02, Hyclone). Transient transfection was performed by transfection of the CHO-S cells with the expression vectors using polyethylenimine transfection reagent (Polysciences, Cat #24765), according to the manufacturer's instructions. Eight to ten days later, the culture supernatants of the transfected cells were harvested and loaded to Protein A Sepharose columns (Bestchrom, Cat #AA0273). The columns were washed with washing buffer (20 mM PB+140 mM NaCl, pH7.4±0.1), and then the recombinant fusion proteins were eluted with eluting buffer (25 mM NaAc+ 100 mM NaCl, pH3.5±0.1). Collected fractions were neutralized with 2 M Tris to pH 5.2±0.2.

### Example 2. Characterization of Recombinant ACVR2A-Fc Fusion Proteins

The recombinant ACVR2A-Fc fusion proteins prepared in Example 1 were measured for protein size and structure in size exclusion-high performance liquid chromatography (SEC HPLC). Briefly, the SEC columns (Xbridge BEH 200 Å, 3.5 μm, 186007640, Waters) were fitted into a HPLC system (ARC-HPLC, Waters) and rinsed with ultrapure water at 0.5 ml/min for 30 min. Then, 5 column volumes (CV) of 50 mM PBS-300 mM NaCl (pH7.3), as the mobile phase, was flowed at a flow rate of 0.5 ml/min for column equilibration, until the baseline become flat, and 50 μg of each fusion protein was drawn by the autosampler, injected into the system, and read for absorbance at 280 nm. The data was analyzed using Empower to determine the peak areas, which were later normalized to calculate the percentages of each component in relation to total components.

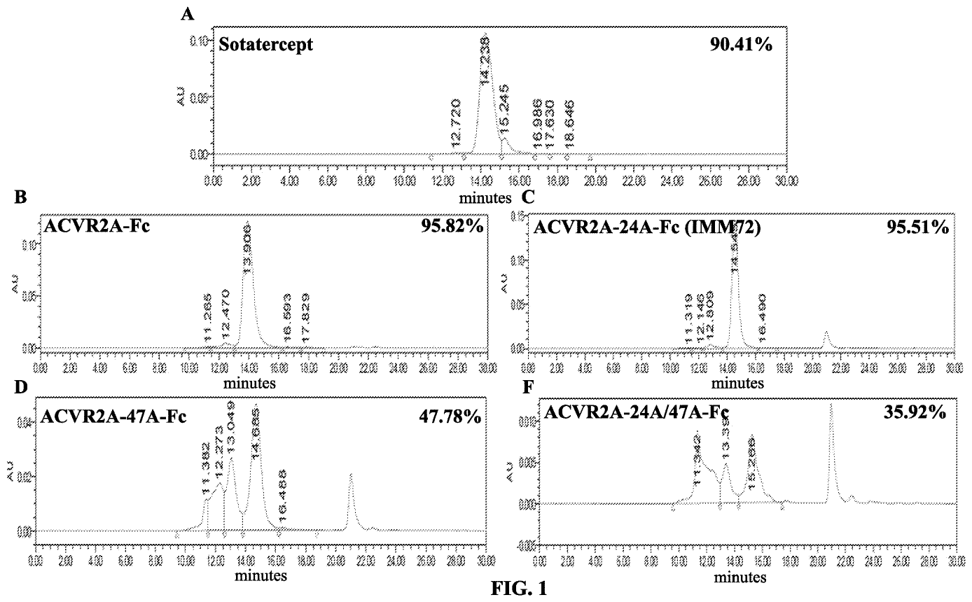

The results were shown in FIG. 1. The main peak for the IMM72 molecule was symmetrical with a narrow peak width (FIG. 1 (C)), indicating the IMM72 molecules were probably homogenous and pure, while there were several peaks for the ACVR2A-47A-Fc and ACVR2A-24A/47A-Fc proteins (FIG. 1 (D & E)), meaning that they may be unstable in structure.

The ACVR2A-Fc fusion proteins were further subjected to reducing SDS-PAGE. Briefly, 10 μl 1 mg/ml each fusion protein diluted in ultrapure water was mixed with 2.5 μl 5× protein loading dye (Cat #: C508320-0001, Sangon Biotech), and incubated at 80° C. for 10 min. The electrophoresis chamber was inserted with a 12% precast polyacrylamide gel (Cat #: C661105-0001, Sangon Biotech) and then added with SDS-PAGE buffer. The fusion protein samples with the loading dye were loaded into the gel wells, respectively. Electrophoresis was performed with the 120 V voltage for 80 min. The gel was then transferred to an automatic staining machine (eStain™ L1, GenScript) for staining and de-staining, with the program set as follows: equilibrium, 1 cycle, 0.5 min; staining, 1 cycle, 0.5 min, and de-staining, 3 cycles, 4 min per cycle. The gel was removed from the staining machine and placed on a white plate for imaging.

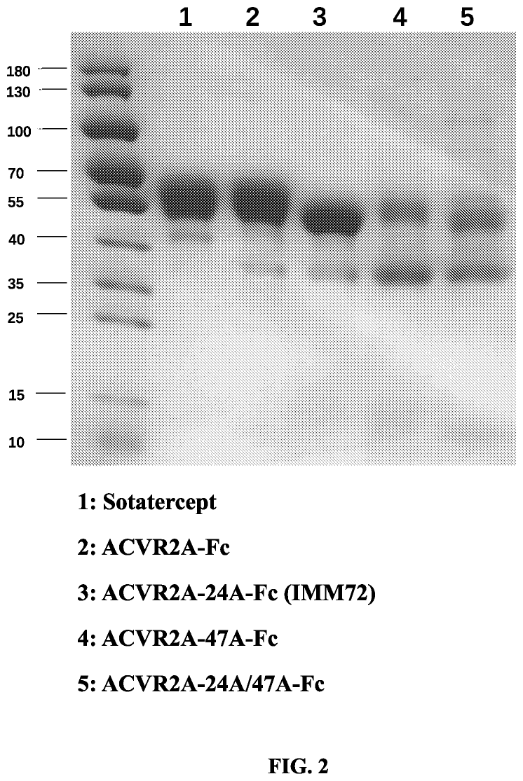

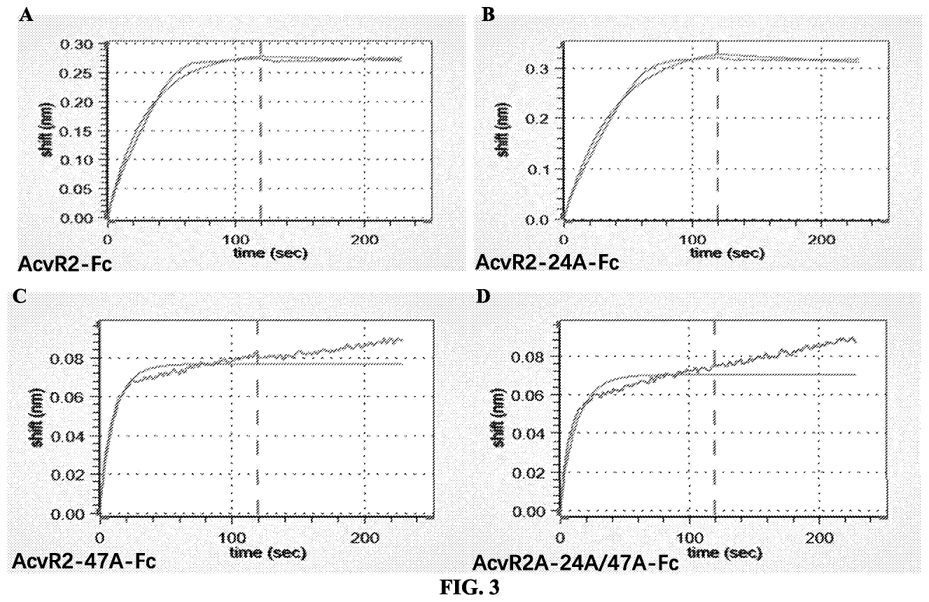

The results were shown in FIG. 2. The main band in lane 3 with IMM72 molecules was clear and narrow, indicating the IMM72 molecules were more homogenous than the ACVR2A-Fc, ACVR2A-47A-Fc and ACVR2A-24A/47A-Fc proteins.

### Example 3. Binding Affinity of Recombinant ACVR2A-Fc Fusion Proteins to Activin A

The binding affinity of the recombinant ACVR2A-Fc fusion proteins to activin A (Cat #: ACV-HM001, Kactus) was measured by bio-layer interferometry (BLI) using a label-free biomolecular detector (Access Medical Systems, Gator bio). The assay employed the Q buffer (10 mM pH7.4 PBS+0.02% Tween+0.2% BSA) and anti-human Fc probe (Cat #20-5036, Gator bio), and the detector was run in the Kinetics Assay mode with each cycle containing loading, association, dissociation and regeneration (detailed program settings set forth in Table 1). The probe was regenerated using the Regeneration buffer (pH 2.0), and the detector was set at 25° C.

TABLE 1
Program setting for label-free biomolecular detector

| | | | Duration |
|---|---|---|---|
| Step Type | Analyte | Concentration | (sec) |
| Baseline | Q buffer | — | 30 |
| Loading | Sample | 10 μg/ml | 60 |
| Baseline | Q buffer | — | 30 |
| Association | Activin A | 0.25 μg/ml | 120 |
| Dissociation | Q buffer | — | 110 |

The data was analyzed using an interaction model (Global, Rmax Unlinked by sensors), and K_{D} values were determined and reported.

The results were shown in Table 2 and FIG. 3. It can be seen IMM72 had a higher dissociation rate constant than the ACVR2A-Fc protein, while ACVR2A-47A-Fc and ACVR2A-24A/47A-Fc showed little or no activin A binding affinity.

TABLE 2
Binding affinity data for ACVR2A-Fc fusion proteins

| | Kₒₙ | | | Loading | | |
|---|---|---|---|---|---|---|
| K_{off} (1/s) | (1/Ms) | KD (M) | Response | Height | Loading | Full R² |
| 0.000257 | 3.20E+06 | 8.03E−11 | 0.274 | 0.8156 | ACVR2A-Fc | 0.986 |
| 0.000455 | 2.51E+06 | 1.81E−10 | 0.32 | 0.9019 | ACVR2A-24A-Fc (IMM72) | 0.988 |
| n/a | 9.10E+06 | n/a | 0.0803 | 0.9037 | ACVR2A-47A-Fc | 0.786 |
| n/a | 7.77E+06 | n/a | 0.0749 | 0.9044 | ACVR2A-24A/47A-Fc | 0.61 |

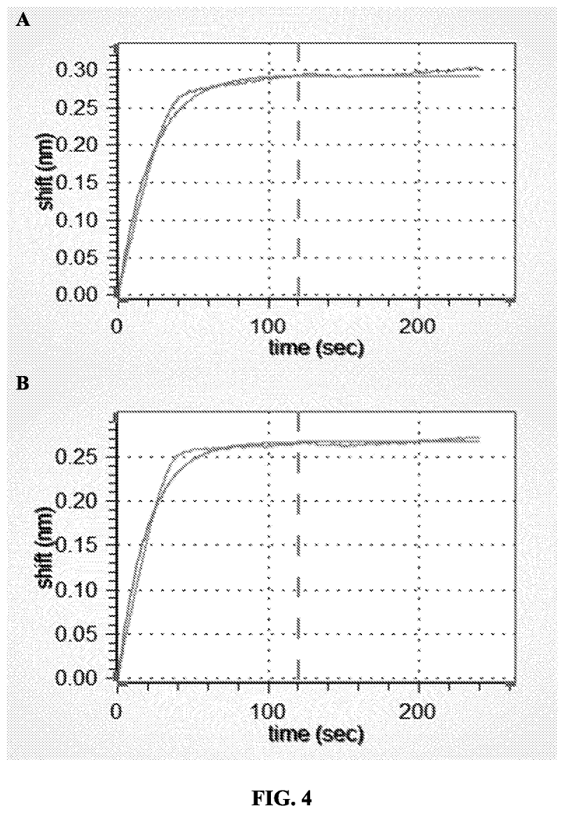

Following the protocol above, IMM72 and the sotatercept analogue were measured for their binding affinity to activin A. The results were shown in Table 3 and FIG. 4.

The two proteins showed similar binding affinity. However, the response at the equilibrium seemed to be higher for the IMM72 molecules, i.e., more activin A molecules might bind to the IMM72 proteins as compared to the sotatercept analogue.

TABLE 3
Binding affinity data for IMM72 and sotatercept analogue

| K_{off} | Kₒₙ | KD | | Loading | | |
|---|---|---|---|---|---|---|
| (1/s) | (1/Ms) | (M) | Response | Height | Loading | Full R² |
| n/a | 4.51E+06 | n/a | 0.293 | 0.8058 | ACVR2A-24A-Fc | 0.987 |
| n/a | 5.07E+06 | n/a | 0.265 | 0.801 | Sotatercept analogue | 0.98 |

### Example 4. Binding Capability of Recombinant ACVR2A-Fc Fusion Proteins to Activin A

The recombinant fusion proteins were tested for their binding capabilities to activin A.

Briefly, an ELISA plate was coated with 100 μl 0.5 μg/mL activin-A proteins (Cat #: ACV-HM001, Kactusbio) in 1×PBS at 2-8° C. overnight, and was blocked with 150 μl PBS with 3% skimmed milk at room temperature for 2 h. Then, the plate was added with 100 μl serially diluted recombinant fusion proteins in PBS with 3% skimmed milk, 3-fold dilution starting at 10 μg/ml, and incubated at 37° C. for 1 h. The plate was washed with 300 μl washing buffer, added with 100 μl Peroxidase-conjugated AffiniPure F(ab′) 2 Fragment Goat Anti-Human IgG, Fc-γ Fragment specific (1:20000 diluted in PBS with 3% skimmed milk, Cat #: 109-036-098, Jackson Immuno), and incubated at 37° C. for 1 h. The plate was washed with 300 μl washing buffer, added with 100 μl TMB (Cat #: 51200050, KPL) and kept in dark. The plate was added with 50 μl 2 M H₂SO₄ to terminate the reaction, and read for the absorbance at 450 nm. The data was analyzed in GraphPad Prism, and the OD450 values were plotted against Log₁₀[protein concentration].

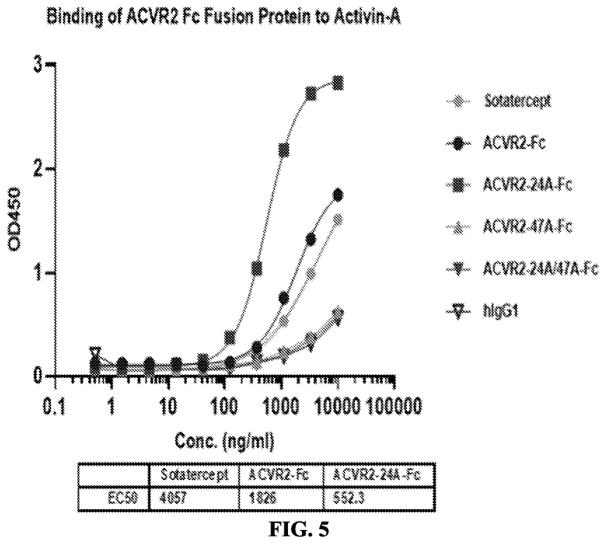

The results were shown in FIG. 5. IMM72 with the N to A mutation at position 24 of SEQ ID NO: 1 showed much higher binding capability to activin A than the sotatercept analogue and the ACVR2A-Fc protein, while the fusion proteins carrying the N to A mutation at position 47 of SEQ ID NO: 2 almost lost their activin A binding capability.

### Example 5. Binding Affinity of Recombinant ACVR2A-Fc Fusion Proteins to Ligands of ACVR2A

The binding affinity of the recombinant ACVR2A-Fc fusion proteins, including the ACVR2A-24A-Fc protein (IMM72), the sotatercept analogue, and the ACVR2A-Fc protein, to recombinant human activin A (Cat #: ACV-HM001, Kactus), recombinant activin B protein (Cat #659-AB-025, R&D systems), recombinant human BMP-10 protein (Cat #2926-BP-025, R&D systems), recombinant GDF-11 protein (Cat #1958-GD-010, R&D systems) and recombinant human GDF-8 (Cat #cyt-418, ProSpec) was respectively measured by bio-layer interferometry (BLI) using a label-free biomolecular detector (Access Medical Systems, Gator bio).

TABLE 4
Program setting for label-free biomolecular detector

| | | | Duration |
|---|---|---|---|
| Step Type | Analyte | Concentration | (sec) |
| Baseline | Q buffer | — | 60 |
| Loading | Sample | 10 μg/ml | 200 |
| Baseline | Q buffer | — | 60 |
| Association | ACVR2A | 2-fold dilution staring at 50 nM | 200 |
| | ligands | (6 concentrations in total) | |
| Dissociation | Q buffer | — | 200 |

TABLE 5
Binding affinity data for ACVR2A-Fc fusion proteins

| Ligand | ACVR2A-Fc Fusion Protein | Koff (1/s) | kon (1/Ms) | KD(M) | Response |
|---|---|---|---|---|---|
| Activin-A | IMC-003 (IMM72) | 7.73E−04 | 1.92E+06 | 4.03E−10 | 0.4897 |
| | Sotatercept | 7.49E−04 | 2.37E+06 | 3.16E−10 | 0.4203 |
| | ACVR2A-Fc (WT) | 8.76E−04 | 2.50E+06 | 3.51E−10 | 0.3861 |
| Activin-B | IMC-003 (IMM72) | 1.13E−03 | 3.48E+05 | 3.25E−09 | 0.6348 |
| | Sotatercept | 1.56E−03 | 4.33E+05 | 3.61E−09 | 0.4765 |
| | ACVR2A-Fc (WT) | 1.79E−03 | 3.88E+05 | 4.62E−09 | 0.4454 |
| BMP-10 | IMC-003 (IMM72) | 1.33E−03 | 6.00E+05 | 2.22E−09 | 0.3627 |
| | Sotatercept | 1.40E−03 | 6.35E+05 | 2.20E−09 | 0.4018 |
| | ACVR2A-Fc (WT) | 1.46E−03 | 6.54E+05 | 2.23E−09 | 0.3768 |
| GDF-11 | IMC-003 (IMM72) | 1.53E−03 | 1.80E+05 | 8.50E−09 | 0.4501 |
| | Sotatercept | 1.61E−03 | 2.20E+05 | 7.33E−09 | 0.3731 |
| | ACVR2A-Fc (WT) | 1.81E−03 | 2.14E+05 | 8.48E−09 | 0.3593 |
| GDF-8 | IMC-003 (IMM72) | 5.84E−004 | 4.69E+004 | 1.24E−008 | 0.1795 |
| | Sotatercept | 8.42E−004 | 6.02E+004 | 1.40E−008 | 0.1732 |
| | ACVR2A-Fc (WT) | 5.36E−004 | 5.56E+004 | 9.65E−009 | 0.1361 |

The assay employed the Q buffer (10 mM pH7.4 PBS+0.02% Tween+0.2% BSA) and anti-human Fc probe (Cat #160003, Gator), and the detector was run in the Kinetics Assay mode with each cycle containing loading, association, dissociation and regeneration (detailed program settings set forth in Table 4). The probe was regenerated using the Regeneration buffer (pH 2.0), and the detector was set at 25° C.

The data was analyzed using an interaction model (Global, Rmax Unlinked by sensors), and K_{D} values were determined and reported.

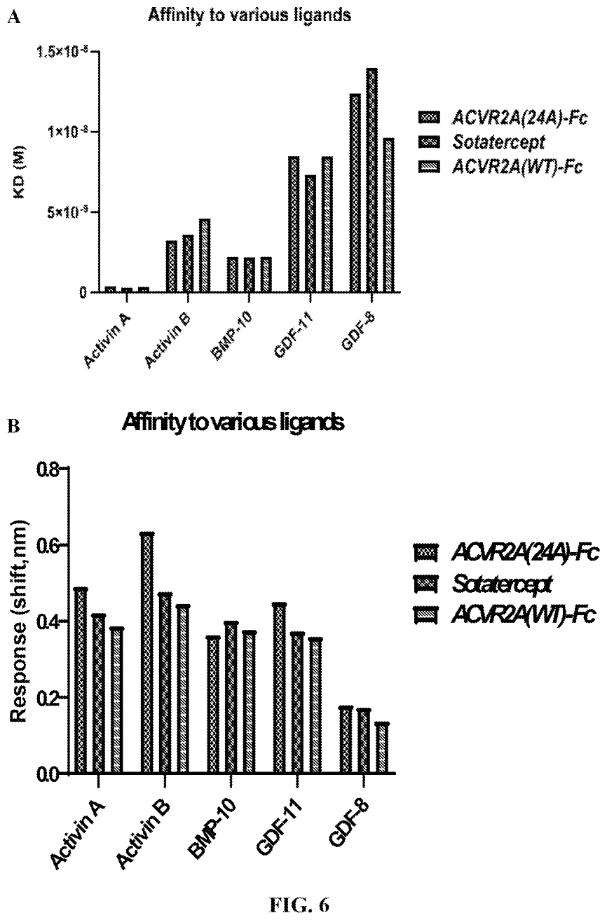

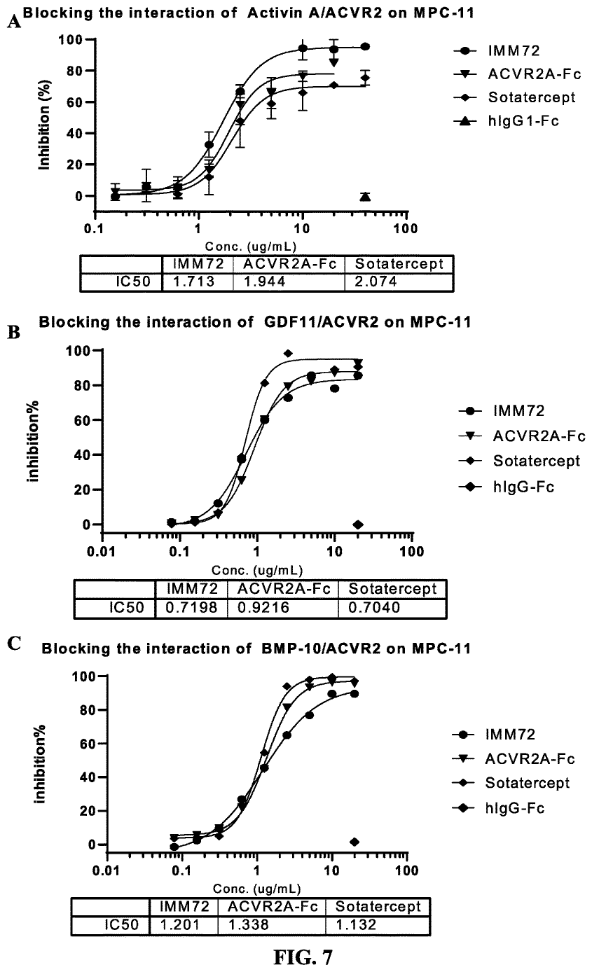

The results were shown in Table 5 and FIG. 6. It can be seen the three recombinant fusion proteins showed similar binding affinity (K_{D}) to each ligand (A). However, the responses at the equilibrium seemed to be higher for the IMM72 molecules, i.e., more ligand molecules might bind to the IMM72 proteins as compared to the sotatercept analogue and the ACVR2A-Fc protein (B).

### Example 6. Capability of Recombinant ACVR2A-Fc Fusion Proteins to Block ACVR2A-Ligand Interaction

The recombinant ACVR2A-Fc fusion proteins, including the ACVR2A-24A-Fc protein (IMM72), the sotatercept analogue, and the ACVR2A-Fc protein, were tested for their ability to block ACVR2A's binding/interaction with its ligands, including activin A, GDF 11 and BMP10.

Briefly, a 96-well plate was added with 50 μl serially diluted IMM72, the sotatercept analogue, or the ACVR2A-Fc protein in DMEM (Cat #12100046, Gibco), 2-fold dilution starting at 120 μg/mL, followed by 50 μl 1.0 μg/mL recombinant human Activin A protein (Cat #ACV-HM001, KACTUS), and then incubated at room temperature for 45 min. The MPC-11 cells (Cat #CBP60951, Co-bioer) were suspended in DMEM at a cell density of 1×10⁵/mL, and 50 μl of such cell suspension was added to each well of the plate. The plate was incubated for 3 days in a 5% CO₂ incubator at 37° C., added with 10 μl CCK-8 solution from Cell Counting Kit-8 (Cat #C0040, Beyotime) and 40 μl DMEM, mixed evenly, and incubated in the incubator for 4 hours. The plate was read for the absorbance at 450 nm in a microplate reader (Tecan, sunrise).

The plate wells added with the MPC-11 cells and the CCK-8 solution were used as the positive control, and the plate wells with the MPC-11 cells, the ligand (activin A) and the CCK-8 solution were used as the blank control. The percent inhibition, calculated as 1−(OD450 test−OD450 Blank)/(OD450 Positive−OD450 Blank)×100%, was used to measure the reduction of MCP-11 cell death and thus the inhibitory effects of the recombinant ACVR2A-Fc fusion proteins on the ACVR2A-activin A binding/interaction.

Following the protocol above, the inhibitory effects of the recombinant ACVR2A-Fc fusion proteins on the ACVR2A's binding/interaction with the other ligands were measured using the recombinant human GDF-11 protein (Cat #1958-GD-010, R&D systems) and the recombinant human BMP-10 protein (Cat #2926-BP-025, R&D systems).

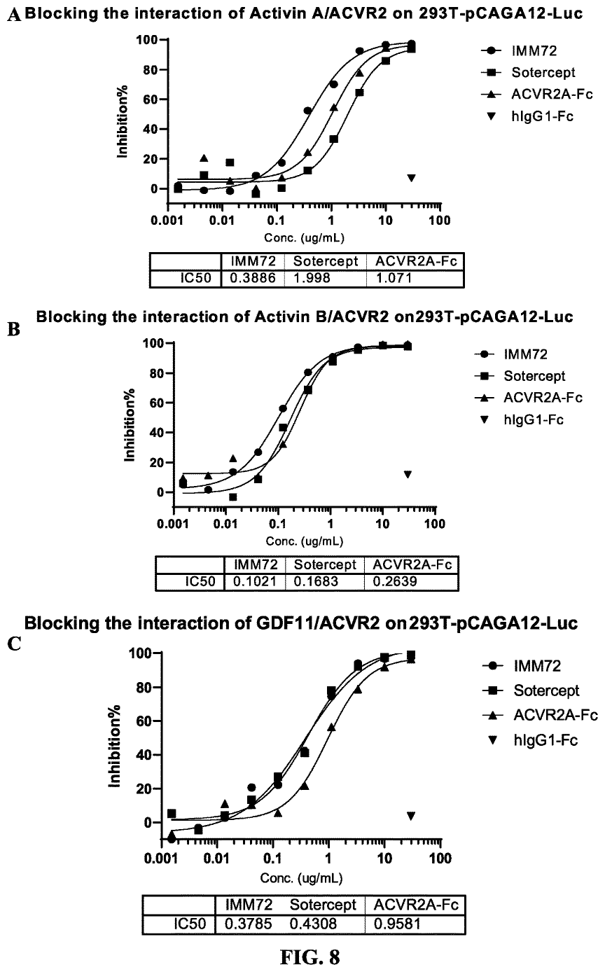

The results were shown in FIG. 7. The IMM72 protein showed higher capability to inhibit ACVR2A-activin A-mediated MPC-11 cell death than the ACVR2A (WT)-Fc protein and the sotatercept analogue (A). The IMM72 protein's inhibitory effects on ACVR2A's binding/interaction with the human GDF-11 protein (B) or the recombinant human BMP-10 protein (C) were almost comparable to that of the ACVR2A (WT)-Fc protein or the sotatercept analogue.

The recombinant ACVR2A-Fc fusion proteins were also tested for their ability to block ACVR2A's binding/interaction with its ligands, including activin A, activin B, GDF 11, BMP10 and GDF8, using the 293T-pCAGA12-Luc cells, prepared by transfection of the 293T cells (Cat #GNHu 43, National Collection of Authenticated Cell Cultures) with pCAGA12-TA-Luc (Cat #D4026-1 μg, Beyotime) plasmid. The interaction of the ACVR2A on the cell surface with its ligands can induce luciferase expression.

Briefly, the 293T-pCAGA12-Luc cells were suspended in DMEM at a cell density of 5×10⁵/mL, and 40 μl of such cell suspension was added to each well of a 96-well nontransparent flat bottom plate. The plate was added with 40 μl serially diluted IMM72, the sotatercept analogue, or the ACVR2A-Fc protein in DMEM (Cat #12100046, Gibco), 3-fold dilution starting at 90 μg/mL, and incubated in a CO₂ incubator for 45 min. The plate was then added with 40 μl 100 ng/ml recombinant human Activin A protein (Cat #ACV-HM001, KACTUS) in DMEM, and then incubated in the incubator overnight. The plate was added with 70 μl substrate solution from Steady-Lumi™ II Firefly Luciferase Assay Kit (Cat #RG059M, Beyotime), and incubated in the incubator for 10 min. The plate was read for the Relative Fluorescence Unit (RFU) in a microplate reader (Molecular Devices, SpectraMax M3).

The plate wells added with the 293T-pCAGA12-Luc cells, the ligand (activin A), and the substrate solution from the Steady-Lumi™ II Firefly Luciferase Assay Kit were used as the positive controls, and the plate wells with the 293T-pCAGA12-Luc cells and the substrate solution from the Steady-Lumi™ II Firefly Luciferase Assay Kit were used as the blank control. The percent inhibition, calculated as 1−(RFU test−RFU Blank)/(RFU Positive−RFU Blank)×100%, was used to measure the reduction of the fluoresce signals from the 293T-pCAGA12-Luc cells and thus the inhibitory effects of the recombinant ACVR2A-Fc fusion proteins on the ACVR2A-activin A binding/interaction.

Following the protocol above, the inhibitory effects of the ACVR2A-Fc fusion proteins on the ACVR2A's binding/interaction with the other ligands were measured using the recombinant human activin B protein (Cat #659-AB-025, R&D systems), the recombinant human GDF-11 protein (Cat #1958-GD-010, R&D systems), the recombinant human BMP-10 protein (Cat #2926-BP-025, R&D systems), and the recombinant human GDF-8 protein (Cat #cyt-418, ProSpec).

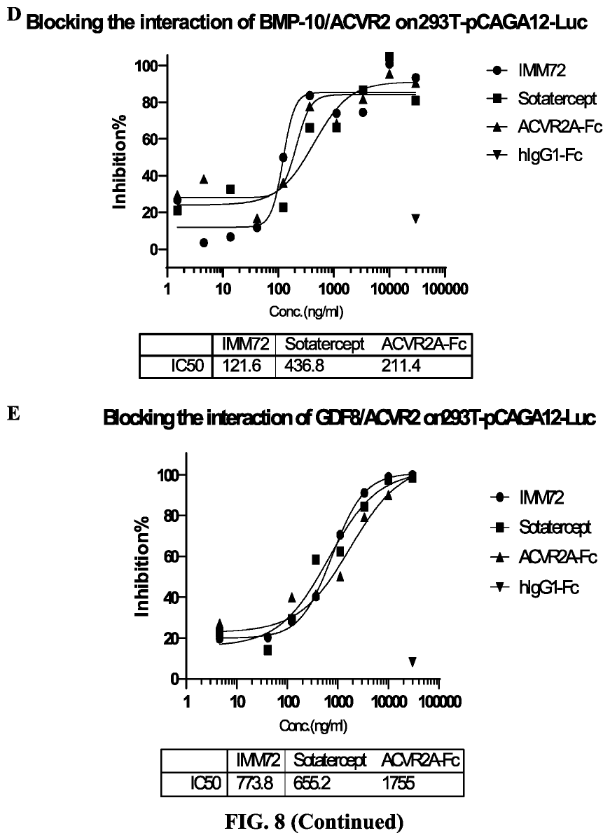

The results were shown in FIG. 8. The IMM72 protein showed much higher capability to inhibit ACVR2A-activin A interaction than the ACVR2A (WT)-Fc protein and the sotatercept analogue (A), and lower IC₅₀ values in blocking ACVR2A-activin B interaction (B), ACVR2A-GDF11 interaction (C) and ACVR2A-BMP-10 interaction (D) than the ACVR2A-Fc (WT) protein and the sotatercept analogue. The IMM72 protein's inhibitory effect on ACVR2A's binding/interaction with the GDF-8 protein was almost comparable to that of the sotatercept analogue (E).

### Example 7. In Vivo Efficacy of Recombinant ACVR2A-Fc Fusion Proteins

The recombinant ACVR2A-Fc fusion proteins were tested for their in vivo efficacy in treating or alleviating pulmonary hypertension in a monocrotaline induced pulmonary arterial hypertension model.

Briefly, 40 male Sprague-Dawley (SD) rats were subcutaneously injected with monocrotaline (MCT), 60 mg/kg body weight, to induce pulmonary hypertension, or the vehicle control (PBS). Seven days later, the rats with MCT treatment were randomly allocated into 3 groups depending on the body weights, and began to receive intraperitoneal (IP) injection of IMM72, the sotatercept analogue or the vehicle control (PBS) according to the scheme in Table 6, and this day was designated as Day 0. The experiment was terminated on Day 23.

TABLE 6
Scheme of animal experiment

| Group | | | |
|---|---|---|---|
| no. | N | Disease induction | Drug administration |
| G1 | 6 | PBS | PBS, i.p. injection, BIW × 3 |
| G2 | 12 | Monocrotaline, | PBS, i.p. injection, BIW × 3 |
| | | 60 mg/kg | |
| G3 | 6 | Monocrotaline, | Sotatercept analogue, 2.5 mg/kg, i.p. |
| | | 60 mg/kg | injection, BIW × 3 |
| G4 | 6 | Monocrotaline, | IMM72, 2.5 mg/kg, i.p. injection, |
| | | 60 mg/kg | BIW × 3 |

The rats were observed for their general appearance (including hair) and activity every day, and recorded for their body weights once per week.

On Day 23, 8, 4 and 5 rats were alive in Groups 2, 3 and 4 respectively. These rats were measured for the pulmonary arterial acceleration time (PAAT), pulmonary arterial ejection time (PET), right ventricular systolic pressure (PVSP), right ventricular fractional area change (RVFAC), tricuspid annular plane systolic excursion (TAPSE) and tricuspid regurgitation peak flow velocity (TRpeak) with the VisualSonics Vevo 2100 imaging system, and then sacrificed to collect the hearts and lungs for further investigation. The heart and lung tissues of the rats died earlier were also collected. The right ventricle (RV), the left ventricle (LV) and the interventricular septum(S) were weighed, and the index of right ventricular hypertrophy (RVHI) was calculated as RV/(LV+S)*100%. The lung tissues were subjected to HE or Masson staining to reveal small vascular dysfunctions.

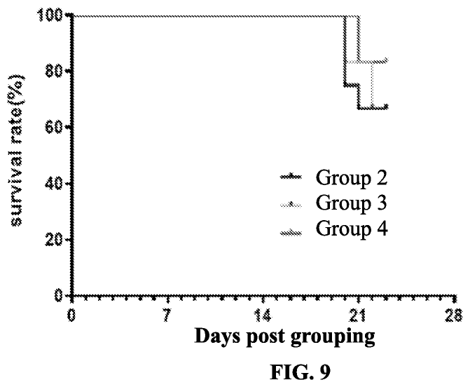

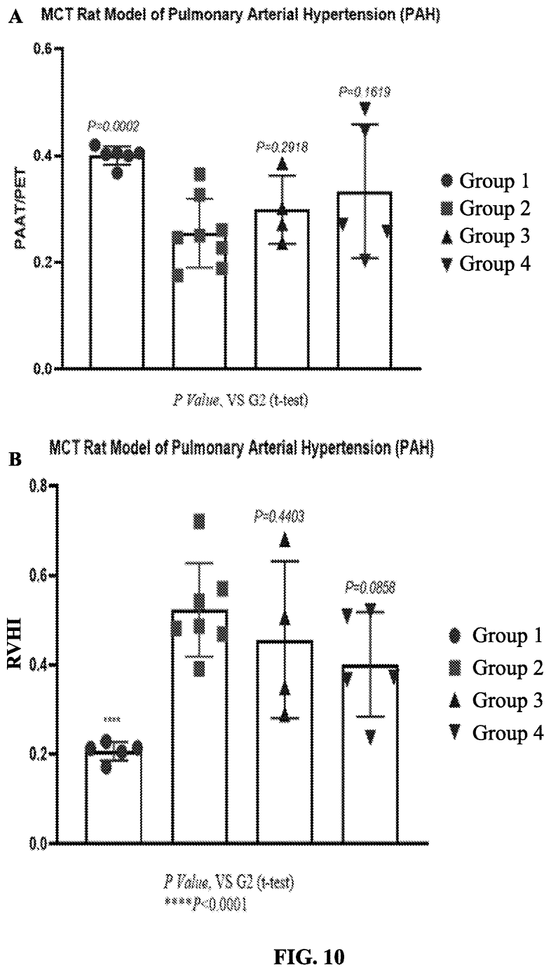

The results were shown in FIG. 9 and FIG. 10.

The rats from Group 2 to 4 that had been injected with monocrotaline had their body weights increased slower than those from Group 1, and the deaths in the three monocrotaline groups were all caused by lung abnormality, e.g., pulmonary congestion and/or respiratory failure, according to the staining results of the lung tissues.

As shown in FIG. 9, the administration of IMM72 or the sotatercept analogue reduced or delayed animal death, as compared to the vehicle control, and the survival rate in the IMM72 administration group was a bit higher that in the sotatercept treatment group.

Further, according to FIG. 10, the administration of IMM72 or the sotatercept analogue slightly increased the PAAT/PET ratio (indicating lower pulmonary artery pressure) (A), and alleviated the right ventricular hypertrophy (B), compared to the vehicle control. The p value was 0.0858 for the t test between Group 2 and Group 4 in terms of right ventricular hypertrophy index, implying a trend to statistically significance.

The sequences of the application were set forth below.

Description/Sequence/SEQ ID NO.
ACVR2A (wildtype and variants)
(SEQ ID NO: 1)
AILGRSETQECLFFNANWEKDRTX1QTGVEPCYGDKDKRRHCFATWKX2ISGSIEI
VKQGCWLDDINCYDRTDCVEKKDSPEVYFCCCEGNMCNEKFSYFPEMEVTQPTS
NPVTPKP
Wildtype ACVR2A
(SEQ ID NO: 1, X1 = N, X2 = N)
AILGRSETQECLFFNANWEKDRTNQTGVEPCYGDKDKRRHCFATWKNISGSIEIVK
QGCWLDDINCYDRTDCVEKKDSPEVYFCCCEGNMCNEKFSYFPEMEVTQPTSNP
VTPKP
ACVR2A variant-24A
(SEQ ID NO: 1, X1 = A, X2 = N)
AILGRSETQECLFFNANWEKDRTAQTGVEPCYGDKDKRRHCFATWKNISGSIEIVK
QGCWLDDINCYDRTDCVEKKDSPEVYFCCCEGNMCNEKFSYFPEMEVTQPTSNP
VTPKP
ACVR2A variant-47A
(SEQ ID NO: 1, X1 = N, X2 = A)
AILGRSETQECLFFNANWEKDRTNQTGVEPCYGDKDKRRHCFATWKAISGSIEIVK
QGCWLDDINCYDRTDCVEKKDSPEVYFCCCEGNMCNEKFSYFPEMEVTQPTSNP
VTPKP
ACVR2A variant-24A/47A
(SEQ ID NO: 1, X1 = A, X2 = A)
AILGRSETQECLFFNANWEKDRTAQTGVEPCYGDKDKRRHCFATWKAISGSIEIVK
QGCWLDDINCYDRTDCVEKKDSPEVYFCCCEGNMCNEKFSYFPEMEVTQPTSNP
VTPKP
Fc domain of human IgG1
(SEQ ID NO: 2)
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSL
SLSPG
Linker
(SEQ ID NO: 3)
GGGGSGGGGSGGGGS
ACVR2A (wildtype or variant)-Fc
(SEQ ID NO: 4)
AILGRSETQECLFFNANWEKDRTX1QTGVEPCYGDKDKRRHCFATWKX2ISGSIEI
VKQGCWLDDINCYDRTDCVEKKDSPEVYFCCCEGNMCNEKFSYFPEMEVTQPTS
NPVTPKPGGGGSGGGGSGGGGSDKTHTCPPCPAPELLGGPSVFLFPPKPKDTLMIS
RTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQ
QGNVFSCSVMHEALHNHYTQKSLSLSPG
ACVR2A-Fc
(SEQ ID NO: 4, X1 = N, X2 = N)
AILGRSETQECLFFNANWEKDRTNQTGVEPCYGDKDKRRHCFATWKNISGSIEIVK
QGCWLDDINCYDRTDCVEKKDSPEVYFCCCEGNMCNEKFSYFPEMEVTQPTSNP
VTPKPGGGGSGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGN
VFSCSVMHEALHNHYTQKSLSLSPG
ACVR2A-24A-Fc
(SEQ ID NO: 4, X1 = A, X2 = N)
AILGRSETQECLFFNANWEKDRTAQTGVEPCYGDKDKRRHCFATWKNISGSIEIVK
QGCWLDDINCYDRTDCVEKKDSPEVYFCCCEGNMCNEKFSYFPEMEVTQPTSNP
VTPKPGGGGSGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGN
VFSCSVMHEALHNHYTQKSLSLSPG
ACVR2A-47A-Fc
(SEQ ID NO: 4, X1 = N, X2 = A)
AILGRSETQECLFFNANWEKDRTNQTGVEPCYGDKDKRRHCFATWKAISGSIEIVK
QGCWLDDINCYDRTDCVEKKDSPEVYFCCCEGNMCNEKFSYFPEMEVTQPTSNP
VTPKPGGGGSGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGN
VFSCSVMHEALHNHYTQKSLSLSPG
ACVR2A-24A/47A-Fc
(SEQ ID NO: 4, X1 = A, X2 = A)
AILGRSETQECLFFNANWEKDRTAQTGVEPCYGDKDKRRHCFATWKAISGSIEIVK
QGCWLDDINCYDRTDCVEKKDSPEVYFCCCEGNMCNEKFSYFPEMEVTQPTSNP
VTPKPGGGGSGGGGSGGGGSDKTHTCPPCPAPELLGGPSVFLFPPKPKDTLMISRT
PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGN
VFSCSVMHEALHNHYTQKSLSLSPG
Sotatercept analogue
(SEQ ID NO: 5)
ILGRSETQECLFFNANWEKDRTNQTGVEPCYGDKDKRRHCFATWKNISGSIEIVKQ
GCWLDDINCYDRTDCVEKKDSPEVYFCCCEGNMCNEKFSYFPEMEVTQPTSNPVT
PKPPTGGGTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNH
YTQKSLSLSPGK

While the application has been described above in connection with one or more embodiments, it should be understood that the application is not limited to those embodiments, and the description is intended to cover all alternatives, modifications, and equivalents, as may be included within the spirit and scope of the appended claims. All referenced cited herein are further incorporated by reference in their entirety.

### REFERENCES

1. Mclaughlin, V. V. et al., (2009). Journal of the American College of Cardiology, 53 (17), 1573-1619.
2. Chin, K. M. et al., (2021). The Lancet Respiratory Medicine, 9 (2), 146-158
3. Ying-Nai Wang, Heng-Huan Lee, Jennifer L. Hsu, Dihua Yu & Mien-Chie Hung (2020) Journal of Biomedical Science, 27, 77
4. Morris F. Tansky et al., (2007) PNAS, 104 (25), 10691-10696
5. Michela Silacci et al., (2014) J Biol Chem. 289 (20), 14392-14398
6. Huili Guo et al., (2017) Mol Biosyst. 13 (3), 598-606
7. Yuelin Kong et al., (2016) Enzyme Microb Technol. 82, 105-109.
8. Marc Humbert et al., (2021) N Engl J Med 384, 1204-1215
9. Marius M. Hoeper et al., (2023) N Engl J Med 388, 1478-1490

1. A recombinant fusion protein, comprising:
i) an extracellular domain of a human activin receptor type 2A (ACVR2A), and
ii) an immunoglobulin heavy chain constant region,
wherein the extracellular domain of the human ACVR2A is linked to the immunoglobulin heavy chain constant region,
wherein the extracellular domain of the human ACVR2A comprises an Asn (N)-to-Ala (A) mutation at a site corresponding to position 24 of SEQ ID NO: 1,
wherein the recombinant fusion protein is able to bind activins and/or growth differentiation factors.

2. The recombinant fusion protein of claim 1, comprising, from N-terminus to C-terminus, the extracellular domain of the human ACVR2A and the immunoglobulin heavy chain constant region.

3. The recombinant fusion protein of claim 1, wherein the extracellular domain of the human ACVR2A comprises an amino acid sequence having at least 95% sequence identity to SEQ ID NO: 1, wherein 24^{th} and 47^{th} amino acid residues in SEQ ID NO: 1 are A and N, respectively.

4. The recombinant fusion protein of claim 3, wherein the extracellular domain of the human ACVR2A comprises the amino acid sequence of SEQ ID NO: 1, wherein 24^{th} and 47^{th} amino acid residues in SEQ ID NO: 1 are A and N, respectively.

5. The recombinant fusion protein of claim 1, wherein the immunoglobulin heavy chain constant region is a human IgG1 or IgG4 heavy chain constant region.

6. The recombinant fusion protein of claim 5, wherein the immunoglobulin heavy chain constant region is an Fc domain of the human IgG1 or IgG4 heavy chain constant region.

7. The recombinant fusion protein of claim 6, wherein the immunoglobulin heavy chain constant region comprises the amino acid sequence of SEQ ID NO: 2.

8. The recombinant fusion protein of claim 1, wherein the extracellular domain of the human ACVR2A is linked via a linker to the immunoglobulin heavy chain constant region.

9. The recombinant fusion protein of claim 8, wherein the linker is a GS linker.

10. The recombinant fusion protein of claim 9, wherein the linker comprises the amino acid sequence of SEQ ID NO: 3.

11. The recombinant fusion protein of claim 1, comprising the amino acid sequence of SEQ ID NO: 4, wherein 24^{th} and 47^{th} amino acid residues in SEQ ID NO: 1 are A and N, respectively.

12. A homodimer of the recombinant fusion protein of claim 1, wherein the homodimer comprises two recombinant fusion proteins linked by one or more disulfide bonds.

13. A nucleic acid molecule encoding the recombinant fusion protein of claim 1.

14. An expression vector comprising the nucleic acid molecule of claim 13.

15. A host cell comprising the expression vector of claim 14.

16. A pharmaceutical composition, comprising the recombinant fusion protein of claim 1, and at least one pharmaceutically acceptable carrier.

17. A method for treating or alleviating pulmonary hypertension or associated complications in a subject in need thereof, comprising administering to the subject a therapeutically effective amount of the pharmaceutical composition of claim 16.

18. a host cell comprising the nucleic acid molecule of claim 13 integrated into its genome.


SEQUENCE LISTING (5 sequences)

SEQ ID NO: 1 - protein, 115 aa, synthetic construct
  VARIANT 24: note = N or A
  VARIANT 47: note = N or A
AILGRSETQECLFFNANWEKDRTXQTGVEPCYGDKDKRRHCFATWKXISGSIEIVKQGCWLDDINCYDRTDCVEKKDSPEVYFCCCEGNMCNEKFSYFPEMEVTQPTSNPVTPKP

SEQ ID NO: 2 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 15 aa, synthetic construct
GGGGSGGGGSGGGGS

SEQ ID NO: 4 - protein, 356 aa, synthetic construct
  VARIANT 24: note = N or A
  VARIANT 47: note = N or A
AILGRSETQECLFFNANWEKDRTXQTGVEPCYGDKDKRRHCFATWKXISGSIEIVKQGCWLDDINCYDRTDCVEKKDSPEVYFCCCEGNMCNEKFSYFPEMEVTQPTSNPVTPKPGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK